(19) United States
(12) Patent Application Publication
(10) Pub. No.: US 2014/0073992 A1
(43) Pub. Date: Mar. 13, 2014

(54) APPARATUS AND METHOD FOR MANUALLY POWERED BODILY FLUID EXTRACTION

(71) Applicants: Kevin A. Hufford, St. Petersburg, FL (US); Hoi-Cheong S. Sun, Tampa, FL (US)

(72) Inventors: Kevin A. Hufford, St. Petersburg, FL (US); Hoi-Cheong S. Sun, Tampa, FL (US)

(73) Assignee: The Charles Stark Draper Laboratory, Inc.

(21) Appl. No.: 13/612,207

(22) Filed: Sep. 12, 2012

(51) Int. Cl. A61B 5/151

(52) U.S. Cl. 600/583

(57) ABSTRACT

Apparatuses and methods are disclosed herein for extracting bodily fluids. The apparatus consists of a housing, a releasable lancet stored in the housing, an inlet flow channel formed in the housing, a mechanically powered vacuum generator, and a storage area formed in the housing. The releasable lancet is released to pierce the skin of the subject to allow fluid to be extracted from the subject. The vacuum generator generates a partial vacuum within the inlet flow channel. After the lancet has pierced the skin of the subject, the partial vacuum draws fluid from the subject and into the inlet flow channel. The storage area is in fluid connection with the inlet flow channel, and receives the fluid drawn from the subject from the inlet flow channel.

### FIELD OF THE INVENTION

In general, the disclosure relates to a manually powered apparatus for extracting and storing a bodily fluid from a subject and a method for using the apparatus to extract and store a bodily fluid.

### BACKGROUND OF THE INVENTION

Blood diagnostic tests in which only a small amount of blood is needed generally involve a two step process. For example, for a finger prick test, a lancet is first used to puncture the skin of a subject's finger so that blood can be extracted. After squeezing the finger to release an adequate amount of blood, a sampler is used to collect the sample from the surface of the subject's finger.

This kind of two-step testing has several drawbacks. First, each step provides an opportunity for user error. If a larger sample is required, there is a chance of spilling or smearing the sample before it is collected by the sampler or while it is being collected. In this case, more blood will need to be extracted from the puncture or a second sample will need to be drawn, causing discomfort to the subject. Furthermore, requiring two separate devices increases the equipment needed and medical waste. The amount of equipment and waste is especially burdensome in mobile testing. In addition, if a precise amount of fluid is needed for a sample, it is difficult to extract the correct amount of fluid using blood lancets and samplers, and additional squeezing or a second finger prick may be needed to draw the amount of fluid needed for the test if the correct amount was not drawn initially.

### SUMMARY OF THE INVENTION

There is therefore a need for a manually powered apparatus that can automatically puncture a subject's skin, extract a bodily fluid, and store the bodily fluid. The apparatus should extract the bodily fluid with minimal pain and inconvenience, and without needing to squeeze the subject's skin. The apparatus should also be able to extract a controlled sample size. Such apparatuses can be used for a wide range of testing, such as blood glucose monitoring, glycated hemoglobin (HbA_{1c}) analysis, blood gas analysis, cholesterol level testing, and protein immune assays. For portability and ease of use, the apparatus should be mechanically powered and should not require a power source.

Accordingly, one aspect of the disclosure relates to a manually powered apparatus for extracting and storing a bodily fluid. The apparatus consists of a housing, a releasable lancet stored in the housing, an inlet flow channel formed in the housing, a mechanically powered vacuum generator, and a storage area formed in the housing. The releasable lancet is released to pierce the skin of the subject to allow fluid to be extracted from the subject. In one implementation, the lancet passes through the inlet flow channel during the release of the lancet. The vacuum generator generates a partial vacuum within the inlet flow channel. After the lancet has pierced the skin of the subject, the partial vacuum draws fluid from the subject and into the inlet flow channel. The storage area is in fluid connection with the inlet flow channel, and receives the fluid drawn from the subject from the inlet flow channel. In one implementation, the apparatus includes a spring for retracting the lancet back into the housing after its release.

In certain implementation, the apparatus includes a septum that forms at least a part of a wall of the inlet flow channel. The septum is positioned such that it is pierced by the lancet during the release of the lancet. A barb on the lancet stretches the septum after release of the septum. The stretching of the septum mechanically generates the partial vacuum used to draw out the bodily fluid.

In certain implementations, the vacuum mechanism includes a deflatable, resilient bladder in fluid connection with the inlet flow channel. Deflating and releasing the deflatable bladder creates the partial vacuum in the inlet flow channel for drawing the fluid into the storage area. In one such implementation, the deflatable bladder includes a flexible membrane engagable by an operator to manually deflate the bladder. In another implementation, the apparatus includes a rotatable cam, which when rotated, cause the deflatable bladder to deflate create partial vacuum. In some such implementations, the cam is further configured such that its rotation triggers the release of the lancet. In one implementation, the deflatable bladder serves as the storage area.

In various ones of the implementations referred to above, the vacuum generator is configured such that the partial vacuum is automatically released after a predetermined amount of fluid has been collected.

In some implementations, the apparatus includes a capillary flow channel in fluid connection with the inlet flow channel and the storage area. The capillary flow channel causes fluid to flow from the inlet flow channel to the storage area via capillary action. In some implementations, the apparatus includes at least one assay channel in fluid connection with at least one of the inlet flow channel and the storage area for running a test on extracted fluid. In some implementations, the apparatus includes a fluid port in fluid connection with the storage area for extracting fluid from the storage area. Alternatively, the storage area may include a pierceable membrane which, when piered, allows the extraction of fluid from the storage area. In some implementations, the apparatus is configured to extract blood. In such implementations, the apparatus may include a separation area in fluid connection with the inlet flow channel and in fluid connection with the storage area for separating blood plasma from the blood. After the blood plasma separates from the blood, the blood plasma flows from the separation area to the storage area.

Another aspect disclosed herein relates to a method for extracting bodily fluids using a fluid extraction apparatus. The method includes, releasing, by the fluid extraction apparatus, a spring-loaded lancet housed therein in response to a manual manipulation of a mechanical trigger incorporated into the fluid extraction apparatus. A partial vacuum is then induced within an inlet flow channel of the fluid extraction apparatus, thereby drawing fluid from a subject through a surface of the subject pierced by the lancet as a result of its release. The fluid drawn by the vacuum is then stored within the fluid extraction apparatus.

According to still another aspect, the disclosure relates to an apparatus for extracting bodily fluids. The apparatus includes a housing and a lancet housed in the housing for piercing the skin of a subject when the lancet is released to allow fluid to be extracted from the subject. The apparatus also includes a manual triggering means for releasing the lancet, an inlet flow channel formed in the housing, and a vacuum means for generating a partial vacuum within the inlet flow channel. The partial vacuum draws fluid from the subject and into the inlet flow channel after the lancet has pierced the skin of the subject. A storage area formed in the housing in fluid connection with the inlet flow channel receives the fluid drawn from the subject.

### BRIEF DESCRIPTION OF THE DRAWINGS

The system and method may be better understood from the following illustrative description with reference to the following drawings in which:

FIG. 1 is a perspective view of an illustrative implementation of an apparatus for piercing the skin of a subject, extracting fluid from the subject, and storing the fluid extracted from the subject;

FIG. 2 is a flowchart of a method for extracting a bodily fluid using the apparatus of FIG. 1, according to an illustrative implementation of the invention;

FIG. 3 is a series of diagrams illustrating a top view of an illustrative implementation of an apparatus for piercing the skin of a subject with a lancet, creating a vacuum when the lancet retracts, extracting fluid from the subject, and storing the fluid extracted from the subject;

FIG. 4 is a flowchart of a method for extracting a bodily fluid using the apparatus of FIG. 3, according to an illustrative implementation of the invention; and

FIG. 5 is a series of diagrams illustrating a top view of an illustrative implementation of an apparatus having a rotating member that, when engaged, causes a lancet to pierce the skin of a subject and deflates a deflatable bladder to create a vacuum within the apparatus for extracting fluid from the subject.

FIGS. 6A-6C are various viewers of a lancet release for triggering the apparatus of FIG. 1, according to an illustrative implementation.

### DESCRIPTION OF CERTAIN ILLUSTRATIVE IMPLEMENTATIONS

To provide an overall understanding of the invention, certain illustrative implementations will now be described, including apparatuses and methods for extracting and storing a bodily fluid from a subject. However, it will be understood by one of ordinary skill in the art that the systems and methods described herein may be adapted and modified as is appropriate for the application being addressed and that the systems and methods described herein may be employed in other suitable applications, and that such other additions and modifications will not depart from the scope thereof.

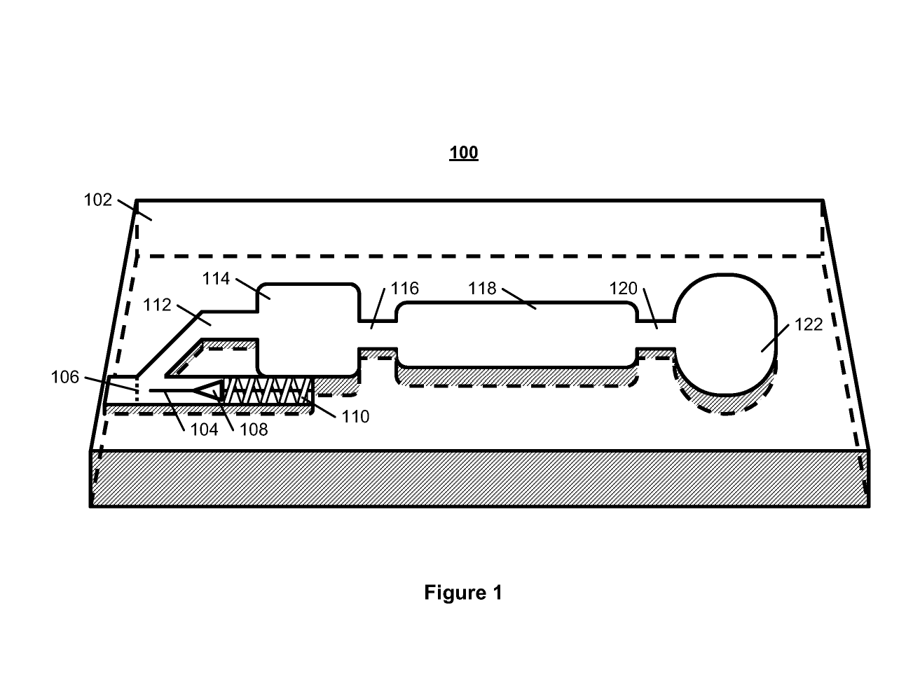

An apparatus for extracting a bodily fluid from a subject using a single, manually powered apparatus is shown in FIG. 1. FIG. 1 is a perspective view of an apparatus 100 that pierces the skin of a subject with a lancet 104, extracts fluid from the subject, and stores the fluid extracted from the subject in a storage area 118. A spring-loaded lancet 104 is released from the apparatus 100 and pierces the skin of the subject, allowing the apparatus 100 to extract a bodily fluid. In some implementations, the bodily fluid is blood. The apparatus can extract blood from, for example, a finger, an arm, or the belly of the subject. In other implementations, the apparatus extracts interstitial fluid by puncturing similar regions of the skin. After the lancet has pierced the subject's skin, the bodily fluid is suctioned into an inlet flow channel 112 by a partial vacuum created within the apparatus 100. The partial vacuum is created manually, e.g., by deforming a deflatable bladder 122 that is in fluid connection with the inlet flow channel 112. The partial vacuum and/or capillary forces within the inlet flow channel 112 draw the bodily fluid into a sample preparation area 114. The sample of bodily fluids may undergo some preparation, such as separation of one or more components of the extracted fluid, a chemical reaction, or the addition of a tag to the bodily fluid for tagging a certain component of the sample. After the sample has been prepared, another partial vacuum and/or capillary force within a transport flow channel 116 draws the bodily fluid into the storage area 118.

The apparatus 100 may be used for a wide range of testing, such as blood glucose monitoring, glycated hemoglobin (HbA_{1c}) analysis, blood gas analysis, cholesterol level testing, and protein immune assays. The apparatus 100 is configured for collecting a minimal quantity of fluid used in a particular test, e.g., in the range of 10 to 100 μL. The amount of bodily fluid collected by the apparatus 100 depends on requirements of the test being performed and may be outside of this range.

The housing 102 of the apparatus 100 is constructed out of any material in which flow channels and storage areas can be molded, such as glass, a polymer, or silicon. For example, the housing 102 can be constructed out a thermoplastic (e.g., polystyrene, polyimide, polyethylene, or polycarbonate), biodegradable polyester (e.g., polycaprolactone (PCL)), a soft elastomer (e.g., polyglycerol sebacate (PGS)), polydimethylsiloxane (PDMS), or poly(N-isopropylacrylamide). The selection of the material depends on the type of test being performed, the physical requirements of the flow channels or sample areas, the type of bodily fluid being collected, and/or other requirements. For example, if the bodily fluid is tested without removing the bodily fluid from the apparatus 100 and optical sensors or a microscope are to be directed at the apparatus 100, a material with particular optical properties can be selected. If fine topographies or small flow channels are desired, materials that can be molded or etched with fine precision are preferrable. In addition, the selected material should not adversely interact with the bodily fluid. Furthermore, if the apparatus 100 contains one or more other substances (e.g., a chemical tag, an anticoagulant, or another reagent) in addition to the bodily fluid being collected, or if such a reagent is added to the storage area 118 or sample preparation area 114, then the selected material should not adversely interact with the other substance. The topography of the housing can be formed using hot embossing, etching, photolithography, injection molding, soft lithography, machining, casting, or other techniques or any combination of techniques.

The housing 102 of the apparatus 100 contains several fluid channels 112, 116, and 120; several fluid storage regions 114, 118, and 122; and a lancet firing region that houses the lancet 104 and a spring 110. These regions and channels are formed within the housing 102 by injection molding, etching, or any other technique or combination of techniques. Once the regions and channels are formed in the housing, the mechanical components within the housing 102 are installed. For example, the lancet 104 is inserted into the lancet firing region and loaded with the spring 110. Any additional mechanical components or other materials, such as chemical tags, anticoagulants, or other substances, are also installed or inserted within the housing 102.

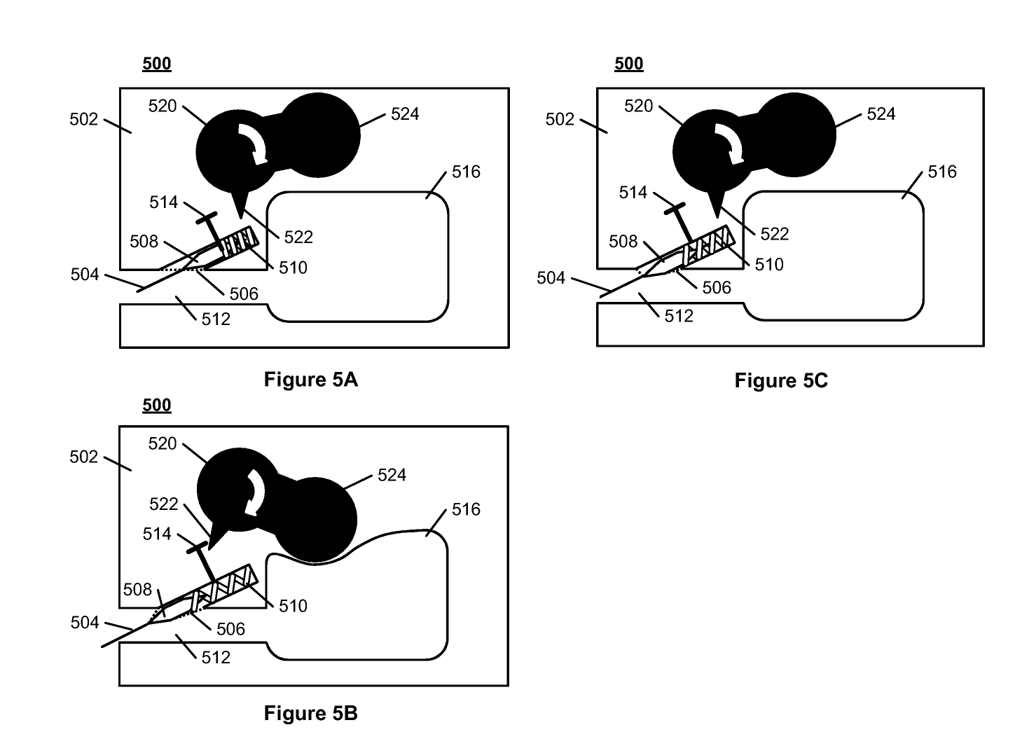

Once the inside of the apparatus 102 has been prepared, the housing 102 is overmolded or bonded to a layer of the same material as the housing 102 or a different material to cover the housing 102 of the apparatus 100. The material selected for the overmold can be based on similar design considerations as discussed above in relation to the housing 102. Furthermore, the overmold layer or a part of the overmold layer can be a pierceable or flexible membrane. For example, the overmold covering the vacuum bladder 122 may be a flexible membrane that forms a wall of the vacuum bladder 122. A user can push down on the flexible membrane that forms the wall of the vacuum bladder 122 to deform the vacuum bladder 122, which causes fluid to exit the vacuum bladder 122. The flexible membrane is resilient, i.e., it returns to its initial shape after the load that has deflected it is removed, thus restoring the vacuum bladder 122 to its original shape and volume and drawing in fluid. If the inlet flow channel 112 is sealed (e.g., by being pressed against the subject's skin) while the flexible membrane is deformed, releasing the flexible membrane would create a partial vacuum within the apparatus 100. The other walls of the vacuum bladder 122 can be formed by the housing 102, but in some implementations, such as the implementation described in relation to FIG. 5, the vacuum bladder 122 additionally has one or more flexible side walls. The lower wall of the vacuum bladder 122 can alternatively or additionally be flexible. In some other implementations, the vacuum bladder 122 is deformed during manufacture of the device, and the resulting vacuum state is maintained by a membrane sealing the inlet channel 112 until the membrane is pierced during usage by the lancet 104.

The storage area 118 can be overmolded with a pierceable membrane so that the storage area 118 can be pierced to extract the bodily fluid. In some implementations, the storage area 118 is the deflatable bladder, and the overmold is both flexible to deform the storage area 118 and pierceable to release the bodily fluid. In some implementations, both the top of the apparatus 100 and the bottom of the apparatus 100 or parts of the top and/or bottom of the apparatus 100 are created by overmolding.

The lancet firing region is a channel for housing a spring-loaded lancet 104. The channel can be cylindrical, rectangular, or another shape. The lancet 104 is a sterile needle for puncturing the skin of a subject. Any suitable lancet gauge may be used, and the gauge can be selected based on the region of skin being punctured, the amount of fluid needed, and/or the preference of the user. A barb 108 is attached to the lancet 104 by overmolding or other means. The lancet firing region also houses a compressed spring 110 which, when released, forces the lancet 104 towards the end of the apparatus 100 so that the tip of the lancet 104 exits the housing 102 of the apparatus 100 to pierce the subject. Thus, the lancet 104 is stored in a retracted position until the compressed spring 110 is released. The release of the spring can be actuated by, for example, a button, a switch, or other manual means. The depth of the lancet that extends from the housing 102 upon release corresponds to the depth of penetration of the subject's skin. The depth of release can be based on the region on the housing that the sample is being taken from and the sensitivity of the subject's skin. For example, the lancet can extend further outside of the housing to pierce a thick part of the skin, such as a fingertip, than to pierce a thinner part of the skin, such as the stomach. The length of the lancet 104 that extends out of the housing 102 upon firing can be adjustable.

After piercing the skin of the subject, the lancet 104 can be retracted again, to its initial position, to a position between its initial position and its fully extended position, or a position further within the housing 102 of the apparatus 100. Preferably, the entirety of the lancet 104 returns to a position fully within the housing 102 of the apparatus 100 after piercing the skin of the subject. This minimizes the possibility that the lancet will come into contact with the subject again or with anyone else. The lancet 104 can be refracted by another spring (not shown) or by a flexible septum 106. If the flexible septum 106 is used to retract the lancet 104, the septum is deflected outward by the barb 108 when the lancet is released by the spring 110. In response to being deflected, the septum 106 pulls back towards its original unflexed position, pulling the barb 108 inward along with the lancet 104. The hole in the septum 106 that is created by the lancet 104 allows the ingress of fluid into the inlet flow channel 112. In addition to retracting the lancet, the septum 106 maintains the sterile interior of the apparatus 110 until it is pierced for collecting the bodily fluid. In some implementations, there is a second septum between the lancet firing region and the inlet flow channel 112 that does not allow for the ingress of fluid. This septum is designed to close tightly around the lancet 104 so that the extracted bodily fluid cannot enter the lancet firing region. Thus, the second septum blocks the lancet firing region from the ingress of fluid, but does not block the inlet flow channel 112. This directs the fluid towards the sample preparation area 114. In some implementations, the second septum is used in place of the septum 106. In this case, the second septum retracts the lancet 104 in a similar manner as the septum 106, and the second septum maintains the sterility of the lancet 104.

The bodily fluid is drawn into the apparatus 100 by a partial vacuum created by the vacuum bladder 122, which is in fluid connection with the inlet flow channel 112 via the sample preparation area 114 and the sample storage area 118. The partial vacuum created within the apparatus 100 draws the bodily fluid into the inlet flow channel 112. In some implementations, the bladder 122 is deformed before the lancet 104 is fired. Air in the bladder can pass through the apparatus 100 and out of the inlet flow channel 112, and after the deflatable bladder 122 has been deformed, the apparatus 100 is pressed against the skin of the subject to create a partial vacuum within the apparatus 100 when the mechanism deforming the bladder (e.g., a user's finger or an internal deflator) is released. The apparatus 100 can be configured to cause the lancet to fire after the bladder 122 has been deformed to remove a predetermined volume of fluid. In other implementations, the partial vacuum is created during or after the lancet 104 has been fired. The outside of the apparatus 100 can have an elastomer coating or other sealing or adhesive material around the end of the inlet flow channel 112 for sealing the apparatus 100 against the skin, which helps maintain the partial vacuum.

In addition to drawing the bodily fluid into the inlet flow channel 112, the partial vacuum can further draw the fluid into the sample preparation area 114, or capillary action within the fluid inlet channel 112 can assist in drawing the extracted fluid into the sample preparation area 114. As described above, sample preparation can include, for example, separating one or more components of the extracted fluid, causing the fluid to undergo a chemical reaction, or adding a tag to the bodily fluid for tagging a certain component of the sample. In some implementations, observations or tests are performed on the bodily fluid in the sample preparation area 114, e.g., to gather baseline data or to determine whether the fluid has been adequately separated, tagged, or otherwise altered. In some implementations, the sample is held in the sample preparation area 114 by a controllable gate, such as a removable barrier or convertible or reversible hydrophobic barrier, until the sample has been prepared. Suitable controllable gates are discussed in U.S. patent application Ser. No. 13/275,859, titled “Apparatus and method for separating plasma from blood and delayed wetting,” incorporated by reference herein in its entirety.

Once the sample has been prepared in the preparation area 114, the sample is drawn though the flow channel 116 and into the sample storage area 118. Capillary action, a partial vacuum created by the deflatable bladder 122, or a combination of capillary action and partial vacuum can be used to transport the fluid into the sample storage area 118. In some implementations, there is no sample preparation area 114, and the extracted bodily fluid flows directly into the sample storage area 118. In some implementations, there is a screen at the end of the sample storage area 118 by the deflatable bladder connection 120 or within the deflatable bladder connection 120 for preventing the fluid from flowing into the deflatable bladder 122. The screen may be constructed of a hydrophobic material or coated with a hydrophobic material so that even if the screen were not fine enough to prevent the flow of fluid into the deflatable bladder 122, the hydrophobic nature of the screen would prevent the extracted fluid from flowing past it and into the deflatable bladder 112.

As mentioned above, capillary action can be use to transport fluid within the apparatus 100 and to assist the vacuum. Capillary action (a.k.a. “wicking”) causes fluid to flow through a channel without any active pumping or other external forces. To create capillary action, any of the flow channels and/or fluid storage regions in the apparatus 100 can be made sufficiently narrow or can be covered by micro-pillars. Suitable micro-pillars can be cylindrical or have elliptical cross sections or polygonal cross sections. The height and diameter of the micro-pillars for promoting capillary action may be in the range of 1-100 μm. The flow rate of the fluid through the fluid inlet channel 112 is controlled by the distance between the micro-pillars. A smaller distance between micro-pillars creates a higher capillary force, which causes the steady state flow rate to be slower. In addition, the longer the fluid inlet channel 112 is, the slower the steady flow rate will be. In some implementations, the micro-pillars create a flow rate in the range of 0.1-10 μL/min. In some implementations, the channels or micro-pillars are coated in a hydrophilic material, such as dextran, to promote capillary action.

In some implementations, tests are performed on the extracted fluid or observations are taken while the fluid is in the sample storage area 118. The apparatus 100 can include observation or analysis equipment, such as optical sensors (e.g., charged-coupled devices (CCDs), photomultipliers, or photodiodes), chemical sensors, or biosensors, or other types of sensors. Alternatively, the apparatus 100 can be positioned in a separate observation or analysis system for observing the extracted fluid without removing it from the sample storage area 118. In some implementations, the apparatus 100 has more than one sample storage area 118. The fluid flowing into the different sample storage areas 118 may be the same or different. For example, different components of the same fluid sample may flow into separate storage areas 118. In some implementations, the apparatus 100 may have separate sample preparation areas 114 to prepare portions of the sample separately. With multiple sample storage areas 118, different tests or duplicate tests can be performed on the extracted fluid.

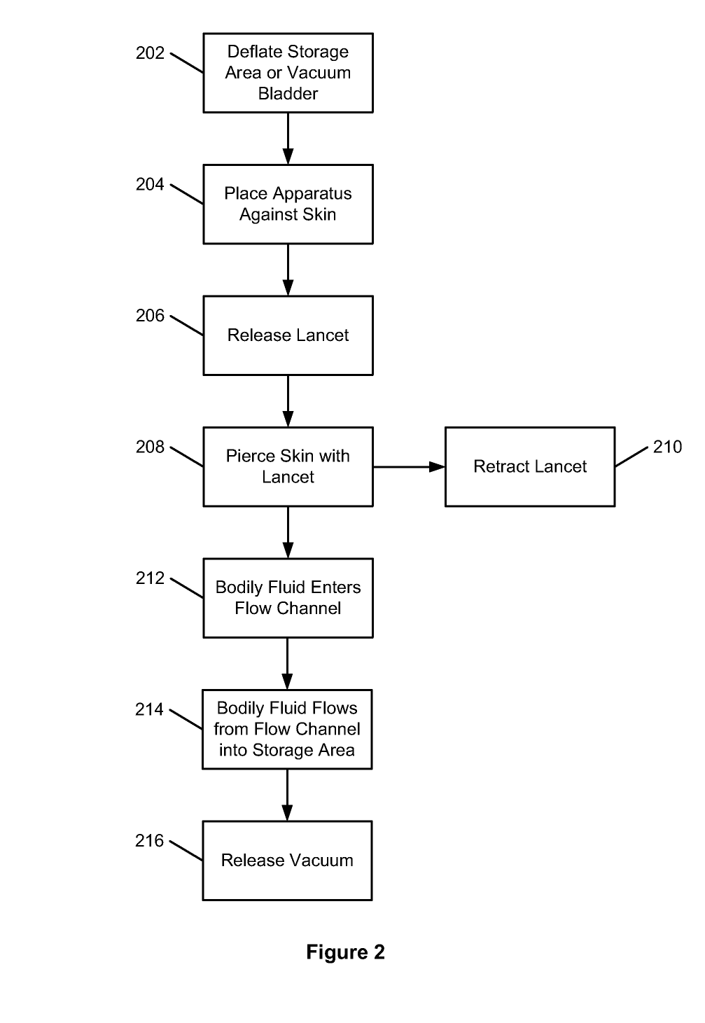

A method of using the apparatus 100 to extract fluid from a subject is shown in FIG. 2. The method involves creating a vacuum within the apparatus 100 (steps 202-204), piercing the skin with the lancet 104 (steps 206-210), and drawing the bodily fluid into the apparatus 100 (steps 212-216).

First, the deflatable bladder 122 described above is deflated (step 202). Alternatively, if the storage area 118 has a flexible membrane or wall, the storage area 118 can be deflated in lieu of using a deflatable bladder 122. The deflatable bladder 122 or storage area 118 can be deformed manually by a user, e.g., by pressing on a wall of the deflatable bladder 122 or storage area 118 with a finger or thumb. In other implementations, the deflatable bladder 122 or storage area 118 can be deformed by a mechanism within the apparatus 100 for deflecting the deflatable bladder 122 or storage area 118 and pushing air out. An example of such a mechanism, a rotatable cam for deflating a storage area, is described in further detail in relation to FIG. 5. In still other implementations, the deflatable bladder 122 is deflated during manufacture of the apparatus 100 such that apparatus 100 maintains the a vacuum state until the lancet 104 is released (step 206).

The deflated apparatus 100 is then pressed against the skin of the subject from whom bodily fluid is being extracted (step 204). When the mechanism deforming the deflatable bladder 122 or storage area 118 is released, the deflatable bladder 122 or storage area 118 tends to return to its initial shape before it had been deformed, thus creating a partial vacuum within the apparatus 100.

After the partial vacuum has been created, the lancet 104 is released (step 206). The spring 110 causes the lancet 104 to be released with enough momentum that it pierces the skin of the subject (step 208). The mechanism that holds the spring in a compressed position is released to allow the spring to extend, pushing out the lancet 104. The mechanism holding the spring in a compressed position can be actuated by a manual user interface, such as a button or a switch; by pressing the apparatus 100 against the subject's skin; or by any other suitable method. After piercing the skin of the subject, the lancet is retracted (step 210). As described above, the lancet 104 can be retracted by a flexible septum 106 that had been deflected by the launch returning to its initial position. Alternatively, an additional spring can be positioned to the side of the barb 108 opposite the spring 110 so that the spring is compressed by the firing of the lancet 104 and extends to push the lancet 104 back towards its initial position.

After the skin has been pierced, the partial vacuum draws bodily fluid from the subject and into the inlet flow channel 112 (step 214). As described above in relation to FIG. 1, the partial vacuum can be supplemented by capillary action within the inlet flow channel 112 to draw the extracted bodily fluid into the sample preparation area 114. Bodily fluid from the subject may continue to be drawn into the inlet flow channel 112 as long as the partial vacuum is maintained.

When a sufficient volume of fluid has been collected, the partial vacuum is released so that the vacuum stops drawing fluid into the inlet flow channel (step 216). The vacuum may be released when all of the displaced air has been replaced by bodily fluid, or the vacuum may be released when a combination of fluid and air replace the displaced air. The amount of air released to create the partial vacuum can be related to the desired volume of fluid to collect. Thus, if a larger sample is desired, a larger volume of air can be evacuated from the apparatus 100. If the deflatable bladder 122 or sample storage area 118 is deformed by the user, the internal structure of the apparatus 100 may permit only a fixed amount of deformation based on the desired volume of fluid. In some implementations, a mechanism within the apparatus can detect whether the desired amount of bodily fluid has been collected. Once the desired amount of bodily fluid has been collected, the apparatus 100 can release the vacuum by allowing the ingress of air through an inlet channel other than inlet fluid channel 112. Alternatively, when enough fluid has been collected, a user can simply remove the apparatus 100 from the subject so that air enters the apparatus 100 through the inlet flow channel 112. In some implementations, the apparatus 100 allows a slow ingress of air while the bodily fluid is being collected. In particular, the septum 106 may permit a slow leak of air along with the bodily fluid; this controls how long the partial vacuum lasts. The speed of air ingress can be related to the desired volume of fluid to collect.

As shown in FIG. 1, the lancet 104 passes through and exits out of the inlet flow channel 112. In this implementation, the apparatus 100 does not need to be moved from its initial position on the subject's skin. In other implementations, the lancet 104 does not pass through the inlet flow channel 112, but rather, may, for example run parallel to the inlet flow channel 112. If such an apparatus were used, a user would move the apparatus after releasing the lancet 104 to align the inlet flow channel 112 with the puncture in the subject's skin. In such implementations, the deflatable bladder 122 or sample storage area 118 may be deformed to create a vacuum after the lancet 104 has punctured the subject's skin.

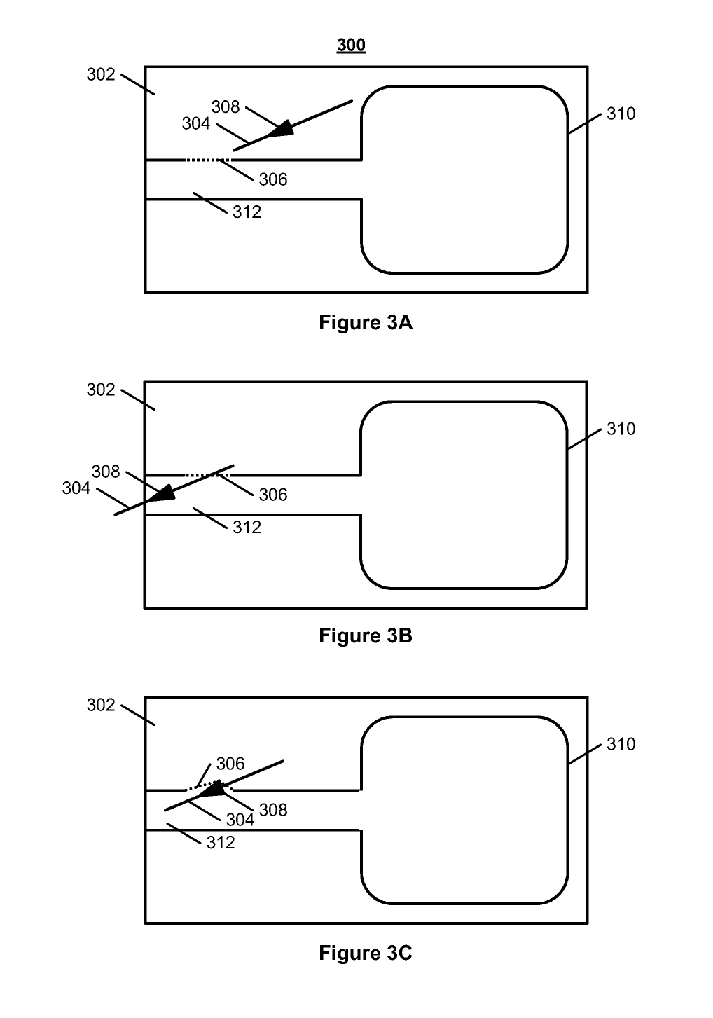

An alternative mechanism for creating a vacuum is shown in FIG. 3. FIGS. 3A through 3C are a series of diagrams illustrating an apparatus 300 in which the partial vacuum is created by retracting the lancet 304 after it pierces the subject. FIG. 3A shows a simplified top view of an apparatus 300 with a housing 302 that has a sample storage area 310 and an inlet flow channel 312 formed therein. The construction of the housing 302 is similar to the construction of the housing 102 of the apparatus 100 of FIG. 1, and the housing can further include a sample preparation area and a lancet firing region as described in relation to FIG. 1. The inlet flow channel 312 has a pierceable septum 306 along a side wall. The lancet 304 is similar to the lancet 104 described in relation to FIG. 1, and the lancet 304 has a barb 308 which is similar to the barb 108 described in relation to FIG. 1. The lancet 304 is positioned at an angle so that it pierces the septum 306 and exits the inlet flow channel 312 to puncture the skin of a subject.

Unlike in FIG. 1, a partial vacuum is not created before the release of the lancet 304. Rather, the apparatus 300 is placed against the skin of a subject without forming a partial vacuum with the skin of the subject. After the apparatus 300 has been positioned against the skin, the lancet 304 is released as shown in FIG. 3B. The lancet 304 is spring loaded in a similar manner as the lancet 104 of FIG. 1. As described above in relation to the lancet 104, the lancet 304 is spring loaded, and a mechanism that holds the spring in a compressed position is released to allow the spring to extend, pushing out the lancet 304. The mechanism holding the spring in a compressed position can be actuated by a manual user interface, such as a button or a switch; by pressing the apparatus 300 against the subject's skin; or by any other suitable method. Upon release, the lancet 304 pierces a hole through the septum 306. The barb 308 also passes through the septum 306 upon release of the lancet 304. The hole formed in the septum 306 tightens around the lancet 304 after the barb has pierced through the septum 306. In an alternative implementation, the barb includes an indentation or other physical stop that prevents it from passing through the septum entirely.

After the lancet has been released, it retracts back into the housing 302 of the apparatus 300 as shown in FIG. 3C. The retraction of the lancet can be caused by a second spring (not shown) or other spring-like member as described in relation to FIG. 1. When the spring retracts, the wide end of the barb 308 catches on the septum 306, and the retraction pulls back the septum 306 from its initial position. The retraction of the 306 increases the volume of the inlet flow channel 312. Since the subject's skin closes the previously opened end inlet flow channel 312, the interior of the apparatus 300 is closed, and the increase in volume creates a partial vacuum within the apparatus 300. This partial vacuum draws the bodily fluid into the inlet flow channel 312.

As shown, the volume created by the movement of the septum 306 is relatively small in relation to the volume of the inlet flow channel 312 and the sample storage area 310. In this case, the partial vacuum begins the flow of bodily fluid into the inlet flow channel 312, and capillary action, as described above in relation to FIG. 1, can be used to transport the bodily fluid through the flow channel 312 and into the sample storage area 310. If a greater amount of evacuation is desired, the septum 306 can be retracted farther. In other implementations, rather than or in addition to deflecting the septum 306, the lancet 304 may be in connection with the sample storage area 310 so that releasing the lancet 304 expands the volume of the sample storage area 310, creating a partial vacuum within the sample storage area 306. Alternatively, the trigger that releases the lancet 304 can cause a different mechanism to expand the volume of the sample storage area 310. Expanding the volume of the sample storage area 310 rather than retracting the septum 306 would prevent bodily fluid from pooling in the cavity created by retracting the septum 306.

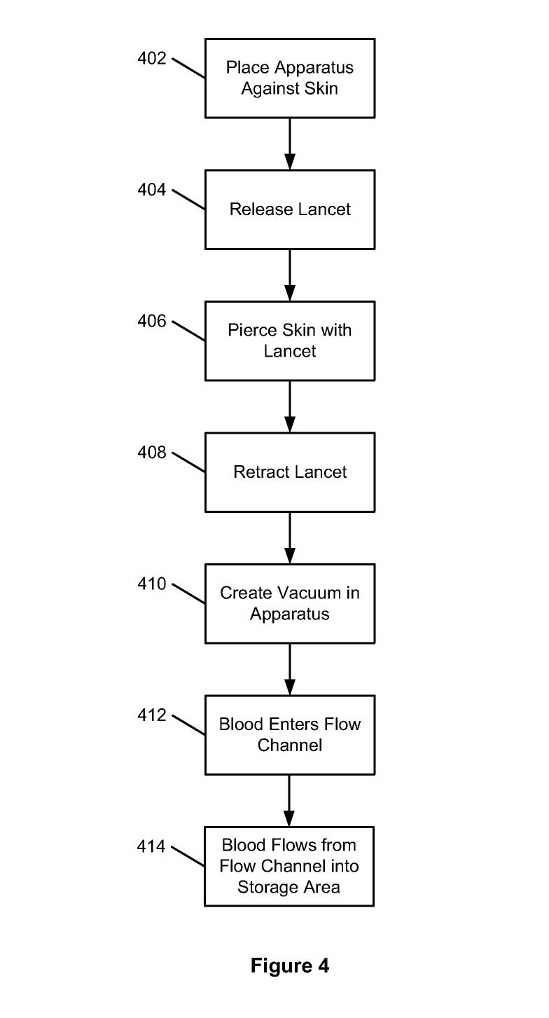

A method of using the apparatus 300 to extract fluid from a subject is shown in FIG. 4. The method involves piercing the skin with the lancet 304 (steps 402-406), creating a vacuum within the apparatus 300 (steps 406-410), and drawing the bodily fluid into the apparatus 300 (steps 412-414).

First, the apparatus 300 is pressed against the skin of the subject from whom bodily fluid is being extracted (step 402). While the sample storage area 310 has not been partially deflated as in the apparatus of FIG. 1, the apparatus 300 should be pressed firmly enough against the skin of the subject so that a seal exists when the partial vacuum is created upon firing the lancet 304. The spring-loaded lancet 304 is then released (step 404). The release can be manually actuated by, for example, pressing a button or turning a switch that causes the release of the spring. The release of the spring ejects the lancet 304 with enough momentum that it pierces the skin of the subject (step 406). After piercing the skin of the subject, the lancet is retracted (step 408). As described above, the lancet 304 can be refracted by a spring or other spring-like mechanism to return the lancet 304 towards its initial position.

When the lancet refracts (step 408), the barb 308 pulls back the septum 306 as shown in FIG. 3C. The retraction of the septum 306 by the barb 308 creates a partial vacuum in the apparatus 300 (step 410). Due to this partial vacuum, bodily fluid from the subject enters the fluid inlet channel 312 (step 412). Once the flow of fluid from the subject and into the channel has begun, capillary action may continue drawing the fluid into the sample storage area 310 (step 414). Alternatively, a secondary vacuum source may be used to aid in drawing the bodily fluid into the sample storage area 310.

In some implementations, the vacuum created by the retracted septum 306 is used for drawing the bodily fluid into a sample preparation area, not shown in FIG. 3. After the sample of bodily fluid has been prepared (e.g., by separating out a component from the sample, or by adding a reagent to the sample), the second partial vacuum or capillary action then draws the fluid from the sample preparation area into the sample storage area 310. The configuration and use of the sample preparation area can be similar to that of the sample preparation area 114 described above in relation to FIG. 1.

Another method for creating the vacuum and retracting the lancet after release is shown in FIG. 5. FIGS. 5A through 5C are a series of diagrams illustrating an apparatus 500 having a rotating member that, when released, deforms a deflatable bladder to create a vacuum within the apparatus 500. FIG. 5A shows a simplified top view of an apparatus 500 with a housing 502 that has a deflatable sample storage area 516, which is also the deflatable bladder, and an inlet flow channel 512 formed therein. The construction of the housing 502 is similar to the construction of the housing 102 of the apparatus 100 of FIG. 1, and the housing can further include a sample preparation area and a lancet firing region as described in relation to FIG. 1. However, in apparatus 500, at least a part of the side wall of the sample storage area 516 is flexible so that the sample storage area 516 can be deformed, as shown in FIG. 5B. In other implementations, the apparatus 500 further includes a deflatable bladder having a flexible side wall; in this case, the side walls of the sample storage area 516 can be rigid. As in FIG. 3, the inlet flow channel 512 has a pierceable septum 506 along a side wall. The lancet 504 is similar to the lancet 104 described in relation to FIG. 1. The lancet 504 is spring-loaded with a compressed spring 510 held in place by a pin 514. The lancet 504 has a barb 508 which may be formed similarly to the barb 108 described in relation to FIG. 1. The lancet 504 is positioned at an angle so that it pierces the septum 506 and exits the inlet flow channel 512 to puncture the skin of a subject.

The apparatus 500 includes a rotating cam 520 for releasing the lancet 504 and deforming the sample storage area 516. The cam 520 rotates about a point in the center of the left circular region of the cam 520, as demonstrated by the arrow drawn on the cam 520. Attached to the cam 520 are a spring release mechanism 522 for releasing the spring 510 and a deflating mechanism 524 for deforming the sample storage area 516. When the cam 520 rotates clockwise, as shown in FIG. 5B, the spring release mechanism 522 moves the pin 514 to release the compressed spring 510. At the same time, the deflating mechanism 524 depresses the wall of the sample storage area 516 so that the sample storage area 516 partially deflates. The cam 520 can be spring-loaded so that it rotates by a fixed amount when a user manually triggers it, e.g., by pushing a button, turning a switch, pressing the apparatus 500 against the subject's skin, or another method. The amount of rotation can be selected based the desired quantity of fluid to be extracted, since the amount of fluid extracted may be related to the amount of deformation of the sample storage area 516 caused by the deflection of its side wall. The cam 520 can be spring-loaded by a torsion spring or a compression spring.

When the lancet 504 is released by the spring 510, the barb 508 pushes against the septum 506 but does not break through the septum 506. In this case, the septum 506 acts as a spring-like mechanism to retract the lancet 504 back towards the lancet firing region, as discussed in relation to FIG. 1. Preferably, the septum 506 retracts the lancet at least back into the flow channel 512, as shown in FIG. 5C, to reduce or eliminate the risk of the lancet accidentally puncturing the subject again or puncturing another person.

After the lancet 504 has been released, the rotating cam 520 rotates counterclockwise to allow the deflatable sample storage area 516 to reinflate, as shown in FIG. 5C. After releasing the lancet 504, the rotating cam 520 can be designed to rotate back to its initial position quickly or slowly. A fast release of the sample storage area 516 may provide a stronger partial vacuum and a faster extraction of fluid. On the other hand, a slower release of the sample storage area 516 may provide a more controlled extraction and more controlled flow of the fluid. As shown, the counterclockwise rotation of the cam 520 does not return the spring 510 or pin 514 to their initial positions.

While FIG. 5 shows the rotating cam 520 directly controlling both the release of the spring 510 and the deflation of the deflatable sample storage area 516, there may be additional or alternative mechanisms within the apparatus 500 for controlling these actions. In some implementations, the lancet 504 is attached directly to or mounted on the rotating cam 520, and rotating the cam moves the lancet 504 outward. In such implementations, the lancet 504 is not separately spring loaded. The rotating cam 520 should be spring loaded so that the lancet 504 exits the apparatus 500 with enough momentum to pierce the skin of the subject.

In operation, the apparatus 500 is placed firmly on the skin of the subject before the partial vacuum is created. When the lancet 504 is released and the deflatable sample storage area 516 is depressed, fluid (i.e., air) in the deflatable sample storage area 516 should exit the deflatable sample storage area 516. However, since the inlet flow channel 512 is blocked by the skin of the subject, the fluid from the inflatable bladder cannot exit via the inlet flow channel 512. So, the deflatable sample storage area 516 can have a fluid escape mechanism (not pictured) through which the fluid in the deflatable sample storage area 516 flows out. The fluid escape mechanism leads either outside of the apparatus 500 or to a second fillable fluid chamber. To prevent the fluid from flowing back into the deflatable sample storage area 516 through the fluid escape mechanism immediately after the vacuum is released, which would eliminate the vacuum created for drawing the bodily fluid, the fluid escape mechanism allows for single-direction fluid flow only. For example, the fluid escape mechanism may have a flap that opens when it experiences outward pressure from the deflatable sample storage area 516, but closes after the partial vacuum has been created.

In some implementations, the fluid escape mechanism allows for a slow, controlled ingress of fluid after the vacuum has been created. The rate with which the partial vacuum is released can be calibrated to the desired amount of bodily fluid to be collected. In other words, the duration during which the apparatus 500 creates a partial vacuum can be controlled by the speed at which fluid is permitted to reenter the apparatus 500 via the fluid escape mechanism or another fluid channel. In such implementations, there can be a deflatable bladder that is separate from the sample storage area 516 so that the controlled ingress of fluid into the deflatable bladder does not contaminate the sample of bodily fluid in the sample storage area 516. In such an implementation, the sample storage area 516 can have hard walls and be in fluid connection with a deflatable bladder. Such a deflatable bladder is similar to the deflatable bladder 122 from FIG. 1, with the addition of the fluid escape mechanism.

If the fluid escape mechanism is part of the sample storage area 516, the fluid escape mechanism can be used to extract the collected bodily fluid from the apparatus for testing. After being collected, the bodily fluid can be released from the deflatable sample storage area 516 by applying pressure to the deflatable sample storage area 516. The application of pressure opens the fluid escape mechanism and allows the fluid to exit. This type of fluid release mechanism may be used in any other apparatus disclosed herein.

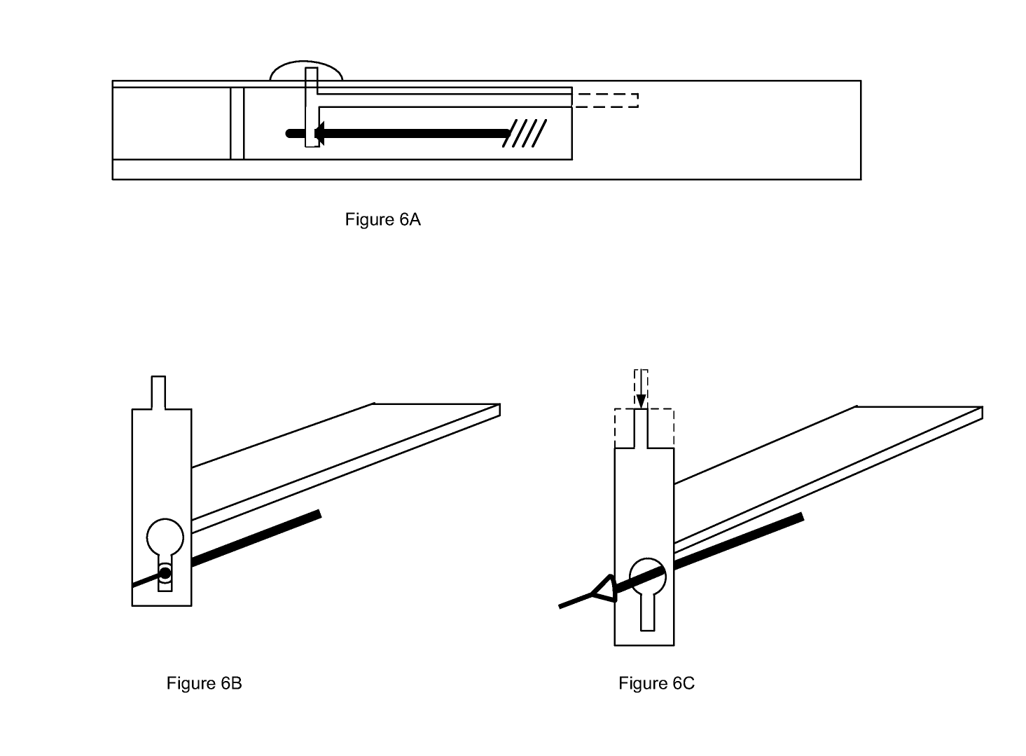

FIG. 6A is a cross-sectional view of the apparatus 100 of FIG. 1, including a lancet release 650 for controlling the release of the lancet 104. FIGS. 6B and 6C show perspective views of the lancet release 650 in un-triggered and triggered positions, respectively.

Referring to FIGS. 6A-6C, the lancet release 650 includes a compliant, but resilient beam 652. The beam 652 includes a vertical portion 654 and a horizontal portion 656. The vertical portion 654 includes a keyhole shaped lancet port 658 and a trigger projection 660. The keyhole shaped lancet port 658 is shaped to have a slot through which the shaft of the lancet 104 can pass, but which is narrow enough to prevent passage of the barb 108. The vertical portion 654 also includes a substantially circular opening (though any suitably sized shape is acceptable) located at the upper end of the slot and sized large enough to allow passage of the barb 108. The trigger projection 660 extends upward through an opening formed in the housing 102 to be adjacent to, or in contact with, a deformable rubber or plastic dome 662 formed over the opening to create a seal. The horizontal portion 656 extends to the back end of the firing region. The far end, opposite the vertical portion 654, is over-molded into the housing 102, supporting the beam in place. In various implementations, the beam 652 can be made from a flexible but resilient metal or plastic, or a combination of the two. For example, the vertical portion may be formed from plastic with a mounting slot for being placed snugly over the end of a metal beam.

In alternative implementations, the lancet release 650 is formed such that it lies alongside the lancet 104 in the firing region, instead of above it. In this implementation, a portion of the side of the housing 102 is removed during manufacturing, to provide access to a user to the trigger projection 660. This portion, along with the distal end of the firing region of the housing 102, can be sealed with a removable adhesive tape or fitted plastic covering to prevent accidental firing of the lancet and to prevent contamination. In either implementation, various safety switches may also be employed to prevent premature triggering of the lancet 104.

In operation, the horizontal portion 656 of the beam 652 maintains the lancet release 650 in the position depicted in FIG. 7B. A user, after placing the apparatus 100 into position against their, or a patient's, skin, depresses the trigger projection 660. The vertical portion 654 shifts downward such that the substantially circular opening aligns with the barb 108, allowing it to pass, thus releasing the lancet 104 through the opening.

While illustrative implementations of the present invention have been shown and described herein, it will be obvious to those skilled in the art that such implementations are provided by way of example only. Numerous variations, changes, and substitutions will now occur to those skilled in the art without departing from the invention. It should be understood that various alternatives to the implementations of the invention described herein may be employed in practicing the invention. It is intended that the following claims define the scope of the invention and that methods and structures within the scope of these claims and their equivalents be covered thereby.

What is claimed is:

1. An apparatus for extracting bodily fluids, the apparatus comprising:
a housing;
a manually releasable lancet housed in the housing for piercing the skin of a subject when the lancet is released to allow fluid to be extracted from the subject;
an inlet flow channel formed in the housing;
a mechanically powered vacuum generator for generating a partial vacuum within the inlet flow channel, wherein the partial vacuum draws fluid from the subject and into the inlet flow channel after the lancet has pierced the skin of the subject; and
a storage area formed in the housing in fluid connection with the inlet flow channel for receiving the fluid drawn from the subject from the inlet flow channel.

2. The apparatus of claim 1, further comprising a septum that forms at least a part of a wall of the inlet flow channel, wherein the septum is pierced by the lancet during the release of the lancet.

3. The apparatus of claim 2, wherein the vacuum mechanism comprises a barb attachment on the lancet, and the barb stretches the septum after the release of the lancet to create the partial vacuum within the inlet flow channel.

4. The apparatus of claim 1, further comprising a spring for retracting the lancet after the lancet has been released.

5. The apparatus of claim 1, wherein the vacuum mechanism comprises a deflatable, resilient bladder in fluid connection with the inlet flow channel, wherein deflating and releasing the deflatable bladder creates a partial vacuum in the inlet flow channel for drawing the fluid into the storage area.

6. The apparatus of claim 5, wherein the deflatable bladder further comprises a flexible membrane that can be engaged by an operator to manually deflate the bladder.

7. The apparatus of claim 5, wherein the vacuum mechanism comprises a rotatable cam configured such that rotating the cam causes the deflatable bladder to deflate to create a partial vacuum within the storage area.

8. The apparatus of claim 7, wherein the cam is further configured such that rotating the cam further causes the release of the lancet.

9. The apparatus of claim 5, wherein the storage area is the deflatable bladder.

10. The apparatus of claim 1, further comprising a capillary flow channel in fluid connection with the inlet flow channel and the storage area, wherein the capillary flow channel causes fluid to flow from the inlet flow channel to the storage area via capillary action.

11. The apparatus of claim 1, further comprising at least one assay channel in fluid connection with at least one of the inlet flow channel and the storage area for running a test on extracted fluid.

12. The apparatus of claim 1, further comprising a fluid port in fluid connection with the storage area for extracting fluid from the storage area.

13. The apparatus of claim 1, wherein the storage area comprises a pierceable membrane which, when pierced, allows the extraction of fluid from the storage area.

14. The apparatus of claim 1, wherein the fluid is blood, the apparatus further comprising a separation area in fluid connection with the inlet flow channel and in fluid connection with the storage area for separating blood plasma from the blood, wherein, after the blood plasma separates from the blood, the blood plasma flows from the separation area to the storage area.

15. The apparatus of claim 1, wherein the vacuum generator is configured such that the partial vacuum is automatically released after a predetermined amount of fluid has been collected.

16. The apparatus of claim 1, wherein the lancet passes through the inlet flow channel during the release of the lancet.

17. A method of extracting bodily fluids by a fluid extraction apparatus, comprising:
in response to a manual manipulation of a mechanical trigger incorporated into the fluid extraction apparatus, releasing, by the fluid extraction apparatus, a spring-loaded lancet housed therein;
mechanically inducing a partial vacuum within an inlet flow channel of the fluid extraction apparatus, thereby drawing fluid from a subject through a surface of the subject pierced by the lancet as a result of its release;
storing the drawn fluid within the fluid extraction apparatus.

18. The method of claim 17, wherein the release of the lancet is after the partial vacuum is induced.

19. The method of claim 17, wherein the release of the lancet is before the partial vacuum is induced.

20. An apparatus for extracting bodily fluids, the apparatus comprising:
a housing;
a lancet housed in the housing for piercing the skin of a subject when the lancet is released to allow fluid to be extracted from the subject;
a manual triggering means for releasing the lancet;
an inlet flow channel formed in the housing;
a vacuum means for generating a partial vacuum within the inlet flow channel, wherein the partial vacuum draws fluid from the subject and into the inlet flow channel after the lancet has pierced the skin of the subject; and
a storage area formed in the housing in fluid connection with the inlet flow channel for receiving the fluid drawn from the subject from the inlet flow channel.
